(19) United States
(12) Patent Application Publication
(10) Pub. No.: US 2011/0270071 A1
(43) Pub. Date: Nov. 3, 2011

(54) MEASURING APPARATUS

(75) Inventor: Yukio Furukawa, Sagamihara-shi (JP)

(73) Assignee: CANON KABUSHIKI KAISHA, Tokyo (JP)

(21) Appl. No.: 13/086,530

(22) Filed: Apr. 14, 2011

(30) Foreign Application Priority Data
Apr. 28, 2010 (JP) 2010-103805

(51) Int. Cl. A61B 6/00

(52) U.S. Cl. 600/407

(57) ABSTRACT

In photoacoustic imaging, in cases where an emitted light amount and a beam pattern of a laser vary according to variation with time and external factors, and cases where the wavelength and the repetition rate of the laser vary, there is a danger that a light fluence of light applied to tissue become too strong. A unit is provided for measuring a distribution of light fluence of light applied to the tissue. A measuring apparatus is provided that controls output of a laser source such that the measured light fluence should not exceed the maximum permissible exposure to the tissue. Accordingly, highly safe photoacoustic imaging to the tissue can be realized.

### BACKGROUND OF THE INVENTION

1. Field of the Invention

The present invention relates to a measuring apparatus, and particularly to a measuring apparatus using a photoacoustic effect.

2. Description of the Related Art

Recently, photoacoustic tomography (PAT) has been proposed, which acquires a distribution of optical characteristics of tissue in a high resolution manner using characteristics of acoustic waves (typically, ultrasound) that scattering in the tissue is little in comparison with light (cf. Japanese Patent Application Laid-Open No. 2005-013597). When the tissue is illuminated with pulsed light generated from a light source, the light diffuses and propagates in the tissue. When an optical absorber included in the tissue absorbs energy of the propagated pulsed light, it generates acoustic waves. Analyzing the acoustic wave signal, a distribution of optical characteristics in the tissue, particularly a distribution of optical energy absorption density, can be acquired.

In the PAT, an acoustic pressure (P) of acoustic waves acquired from the optical absorber of the tissue by optical absorption can be expressed according to a following expression.

P=Γ·μₐ·φ [Expression 1]

where Γ is the Grueneisen coefficient that is an elastic characteristics value, and acquired by dividing a product of the squares of a coefficient of volumetric expansion (β) and a sonic speed (c) by a specific heat (Cp). μₐ is an absorption coefficient of the optical absorber, and Φ is an amount of light in a local region (an amount of light applied to the optical absorber).

An acoustic pressure, which is an acoustic wave signal in the PAT, is proportional to an amount of local light reaching the optical absorber. Since light illuminated on the surface of the tissue is rapidly attenuated in the body owing to scattering and absorption, the acoustic pressure of acoustic waves generated in deep tissue in the body is largely attenuated depending on a distance from a light illumination region. Thus, it is required to increase the amount of illumination light on the surface of the tissue in order to acquire a strong signal.

On the other hand, from the view point of safety of human tissue, in a case of using a laser as a light source, the maximum value of light fluence (amount of illumination illuminated light per unit area) to be illuminated on the human tissue should be kept not to exceed the maximum permissible exposure (MPE) specified by laser safety standards (JIS C6802 and IEC 60825-1).

Japanese Patent Application Laid-Open No. 2008-079835 proposes a system that causes an optical detector to monitor transmitted and scattered light from tissue when the tissue is illuminated with light having a plurality of wavelengths and analyzes the signal, to thereby determine the type of material of a specific site in the tissue.

### SUMMARY OF THE INVENTION

As described above, in the viewpoint of safety of the tissue, it is required to keep the light fluence illuminated on the surface of the tissue not to exceed the MPE. Japanese Patent Application Laid-Open No. 2005-013597 describes that “The light fluence should be equal to or smaller than the maximum permissible exposure (MPE).” However, the description is silent about how to keep the light fluence equal to or smaller than the MPE. More specifically, the cases where, the emitted light amount and the beam pattern of a laser have been changed owing to variation with time and external factors, and cases where the wavelength and the repetition rate of the laser have varied, are not mentioned therein.

An optical detector in Japanese Patent Application Laid-Open No. 2008-079835 monitors transmitted and scattered light from tissue. That is, the detector does not monitor an amount of light itself illuminated on the surface of tissue, and does not consider the MPE. An optical energy adjustment element in Japanese Patent Application Laid-Open No. 2008-079835 is used for adjusting an amount of light having a plurality of wavelengths. The amount of transmitted and scattered light is dependent on a subject. Accordingly, it is difficult to adjust the amount of light from a light source so as to be equal to or smaller than the MPE with reference to a value monitored by the optical detector.

In order to solve the above problems, according to the present invention, a measuring apparatus includes a laser source generating light, a unit for light illumination illuminating tissue with the light, and an acoustic wave detector detecting an acoustic wave generated by the light applied to the tissue, and further includes a unit for detecting optical energy that detects an light fluence of the light onto the tissue, wherein an emitted light amount from the laser source is controlled such that the light fluence detected by the unit for detecting optical energy does not exceed a maximum permissible exposure.

As to the measuring apparatus using a photoacoustic effect, even in cases where an amount of light, a beam pattern, a wavelength and a repetition rate of laser light applied to the tissue vary, an apparatus can be provided that can suppress the light fluence onto the tissue to the MPE or less and thereby is highly safe.

Further features of the present invention will become apparent from the following description of exemplary embodiments with reference to the attached drawings.

### BRIEF DESCRIPTION OF THE DRAWINGS

FIG. 1 is a diagram illustrating a first example.

FIGS. 2A and 2B are diagrams illustrating an operation of the first example.

FIGS. 3A and 3B are diagrams illustrating distribution of light fluence of the first example.

FIG. 4 is a diagram illustrating a second example.

FIGS. 5A and 5B are diagrams illustrating a third example.

FIG. 6 is a diagram illustrating a fourth example.

FIG. 7 is a diagram illustrating a fifth example.

FIG. 8 is a diagram illustrating a sixth example.

FIG. 9 is a diagram illustrating a seventh example.

### DESCRIPTION OF THE EMBODIMENTS

Preferred embodiments of the present invention will now be described in detail in accordance with the accompanying drawings.

According to the laser safety standards (JIS C6802 and IEC 60825-1), in a case of a pulse width of 1-100 nsec, the maximum permissible exposure MPE per pulse to skin is defined by the smaller one of following Expressions (a) and (b).

(a)

E_{MPE}=20 where λ=400-700 nm

E_{MPE}=20·100^{0.002(λ-700)} where λ=700-1050 nm

E_{MPE}=100 where λ=1050-1400 nm [Expression 2]

(b)

E_{MPE}=1100·f⁻¹·t^{-0.75} where λ=400-700 nm

E_{MPE}=1100·10^{0.002(λ-700)}·f⁻·t^{-0.75} where λ=700-1050 nm

E_{MPE}=5500·f⁻¹·t^{-0.75} where λ=1050-1400 nm [Expression 3]

where the unit is mJ/cm², λ is a wavelength (unit: nm). t is a laser illumination time (time period from a start of illumination of light to the finish thereof, unit: second), f is a repetition rate (unit: Hz). More specifically, provided that the measurement time is ten seconds, in cases where the repetition rate is equal to or less than 10 Hz, Expression (a) is applied, and, in cases where the repetition rate is at least 10 Hz, Expression (b) is applied.

The size of aperture used for measuring an amount of light is specified by the laser safety standards (JIS C6802 and IEC 60825-1). In a case of illuminating skin with light having a range of spectrum of 400-1400 nm used for the PAT, the definition is made according to an amount of light measured through an aperture having a diameter of 3.5 mm. This is a standard for averaging by area, set because a light beam does not have a distribution with a uniform amount of light but typically has a certain distribution instead. In a case where the illumination area is larger than a circle with the diameter of 3.5 mm, if an light fluence is acquired by averaging the entire amount of illumination light with respect to the illumination area and the upper limit value of energy per pulse is determined based on the value, since light beam has the distribution of the amount of light, a beam with an amount of light partially exceeding the MPE may be applied. Accordingly, it is required to determine the upper limit of energy per pulse in consideration of the wavelength, rate and measurement time of a laser to be used, and distribution of light actually applied to tissue.

The present invention actually measures a distribution of light fluence of light applied to tissue, and adjusts an amount of light of a laser source such that the maximum value thereof should not exceed the maximum permissible exposure per pulse. Further, the present invention actually measures the repetition rate of the series of pulses and the wavelength of light, sets the maximum permissible exposure per pulse based on the values, and adjusts the amount of light of the laser source.

There is a possibility that the output and wavelength of the laser source may be changed due to variation with time and external factors. There is also a possibility that optical parts in use, such as lenses and mirrors, change in quality due to long time of laser light illumination, and the amount of light and the beam pattern of the laser light applied to the tissue change from the initial conditions. Further, in a case of using a pulse laser source with a passive Q-switch, there may be cases where the temperature and variation with time of the crystal vary the repetition rate and the optimal rate. The present invention can provide an apparatus that is safe for tissue even in such cases.

### EXAMPLES

More detailed configuration will be described in following Examples.

### Example 1

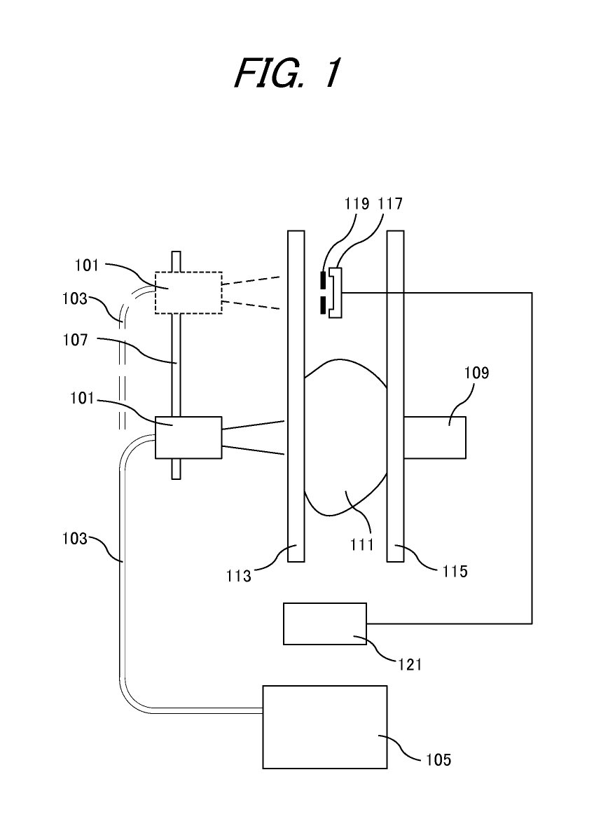

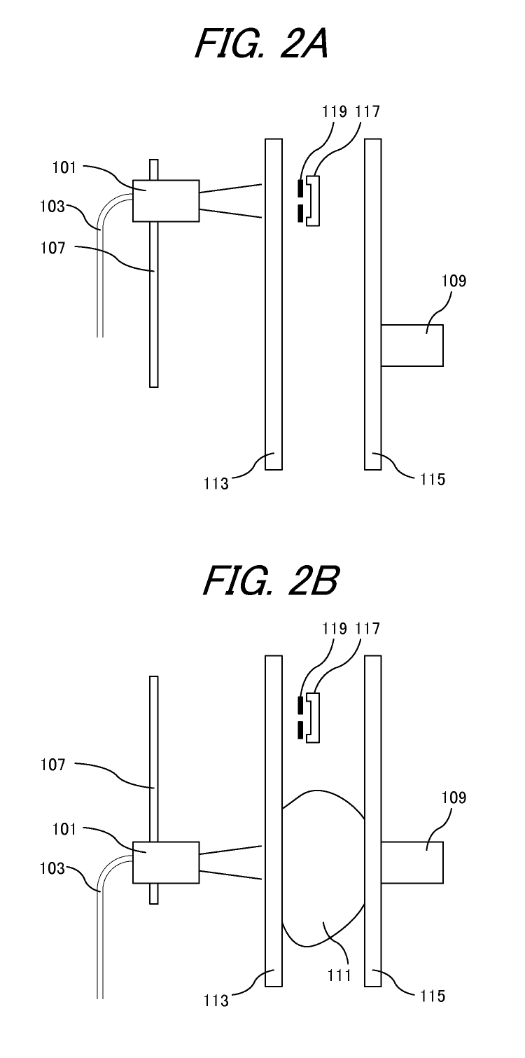

FIGS. 1, 2A and 2B are schematic diagrams illustrating an example of the present invention. In the diagrams, a Nd: YAG laser source 105 generates pulsed light having a wavelength of 1064 nm, a pulse width of 10 nsec and a repetition rate of 10 Hz. A unit 103 for light transmission is configured to include optical fibers. Unit 101 is a unit for light illumination. Acoustic wave detectors 109 are arranged in an array. Tissue 111 may be a mamma of a woman. Supporting plates 113 and 115 support the tissue 111. An aperture 119 is arranged before an optical energy detector 117, and has a through-hole with a diameter of 3.5 mm. The optical energy detector 117 and the aperture 119 configure a unit for detecting optical energy of the present invention. An optical energy display unit 121 displays optical energy and a repetition rate detected by the optical energy detector 117.

The unit 101 for light illumination is equipped on a moving mechanism 107, and capable of being moved in two-dimensional directions parallel to the supporting plate 113.

In this example, the optical energy detector 117 is fixed at a position that does not interfere with holding the tissue and that is equivalent to the tissue, in the measuring apparatus. The position equivalent to the tissue means a position where the unit 101 for light illumination is movable so as to be opposed to the optical energy detector 117 and actually moves to be opposed thereto, and where the distance from the unit 101 for light illumination corresponds to the distance between the unit 101 for light illumination and tissue 111. When an light fluence is measured, the moving mechanism (second moving mechanism) moves the unit 101 for light illumination to the position opposed to the optical energy detector 117 (FIG. 2A). The moving mechanism 107 (first moving mechanism), which is a driving mechanism, then two-dimensionally scans with the unit 101 for light illumination, thereby measuring a distribution of optical energy having passed through the aperture 119. The measured optical energy is divided by the aperture area, thereby a distribution of light fluence is acquired. Information, such as the measured value and distribution of light fluence, is displayed on the optical energy display unit 121. A configuration may be adopted where the first moving mechanism for two-dimensionally scanning with the unit 101 for light illumination and the second moving mechanism for moving the unit 101 for light illumination to the position opposed to the optical energy detector 117 are operated by a common moving mechanism 107. Instead, a configuration may be adopted where the first and second moving mechanisms are operated by respective units different from each other.

In a case where the maximum value of the distribution of light fluence exceeds the maximum permissible exposure per pulse, the emitted light amount of the laser source 105 is adjusted such that the maximum value of the distribution of light fluence becomes equal to or smaller than the maximum permissible exposure per pulse. After such adjustment, the tissue is illuminated with light and information of the tissue is acquired (FIG. 2B).

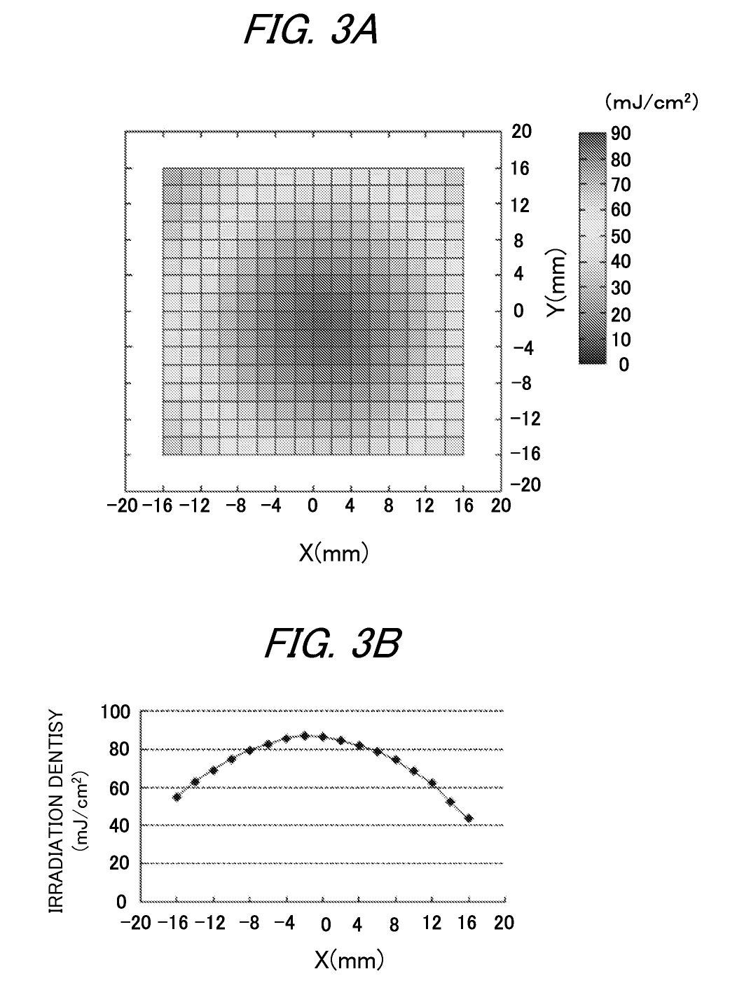

FIG. 3A and FIG. 3B illustrate distribution of light fluence after adjustment of the emitted light amount of the laser source 105. FIG. 3A is a two-dimensional light fluence map. FIG. 3B illustrates a distribution at a peak position (y=−2 mm). Although the maximum permissible exposure per pulse under conditions of this example is 100 mJ/cm², it can be understood that the peak is suppressed to about 90 mJ/cm² according to FIG. 3B.

This example enables the emitted light amount from the laser source 105 to be adjusted such that the light fluence preliminarily becomes equal to or smaller than the maximum permissible exposure before the tissue is actually illuminated with light. Accordingly, a highly safe apparatus can be provided.

In this example, the optical energy distribution is measured by two-dimensionally scanning the unit 101 for light illumination. However, a driving mechanism (first moving mechanism) capable of two-dimensionally scanning may be provided on an optical energy detector side. In this case, the measuring apparatus may employ a configuration capable of scanning only by one of the unit 101 for light illumination and the optical energy detector 117. Instead, the apparatus may employ a configuration capable of scanning by both.

### Example 2

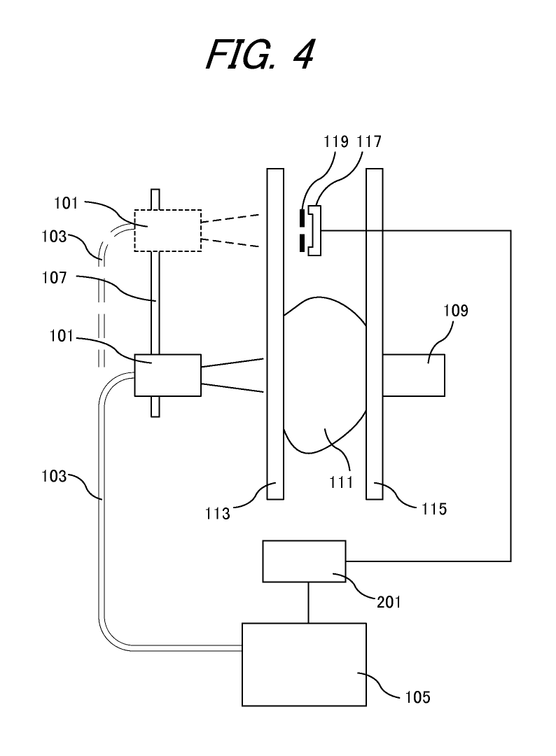

FIG. 4 is a schematic diagram illustrating a second example of the present invention. In this diagram, elements identical to those in FIG. 1 are assigned with the identical numerals. The description thereof is omitted. The difference from the first example is in that the unit 201 for controlling optical energy, which determines the optimal output of the laser source based on the optical energy distribution and the repetition rate measured by the optical energy detector 117, is provided.

As with Example 1, the optical energy distribution is preliminarily measured before measurement of the tissue, and the distribution of light fluence is acquired. In this example, a Nd: YAG laser is employed as the laser source, and the wavelength is known.

The unit 201 for controlling optical energy calculates the maximum permissible exposure per pulse from the wavelength, the repetition rate and the measurement time, and compares the maximum permissible exposure and the maximum value of the measured distribution of light fluence with each other. When the maximum value of the distribution of light fluence exceeds the maximum permissible exposure, the unit 201 controls the laser source 105 such that the output thereof should be equal to or smaller than the maximum permissible exposure. When the maximum value of the distribution of light fluence is smaller than the maximum permissible exposure, the unit 201 causes the laser source 105 to increase the output thereof in an extent of a desired safety factor. The measurement time is an item appropriately set by an operator.

In this example, the output of the laser source is automatically adjusted, thereby improving the operability.

In this example, the unit 201 for controlling optical energy calculates the maximum permissible exposure per pulse from the wavelength, the repetition rate and the measurement time, which may preliminarily be stored in a lookup table instead.

### Example 3

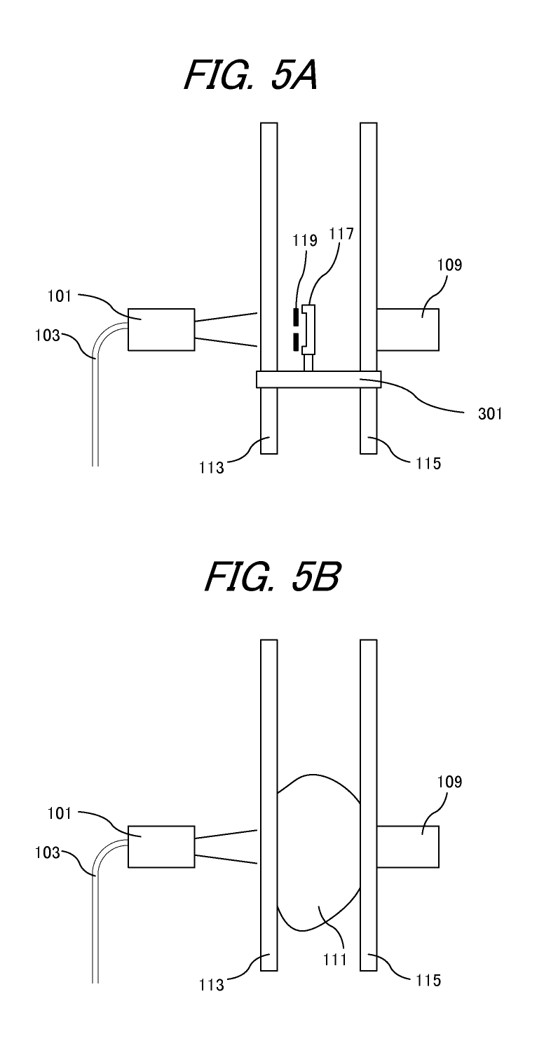

FIGS. 5A and 5B are schematic diagrams illustrating a third example of the present invention. In this diagram, elements identical to those in FIG. 1 are assigned with the identical numerals. The description thereof is omitted. The difference from the first example is in that the optical energy detector 117 is fixed to the fixing part 301 and detachable.

In this example, when the light fluence is measured, the optical energy detector 117 is arranged at a position substantially identical to that for holding the tissue as illustrated in FIG. 5A. When the tissue is measured, the optical energy detector 117 is detached (FIG. 5B).

In this example, the position of the tissue and the position for measuring the light fluence are substantially identical to each other, thereby improving accuracy.

### Example 4

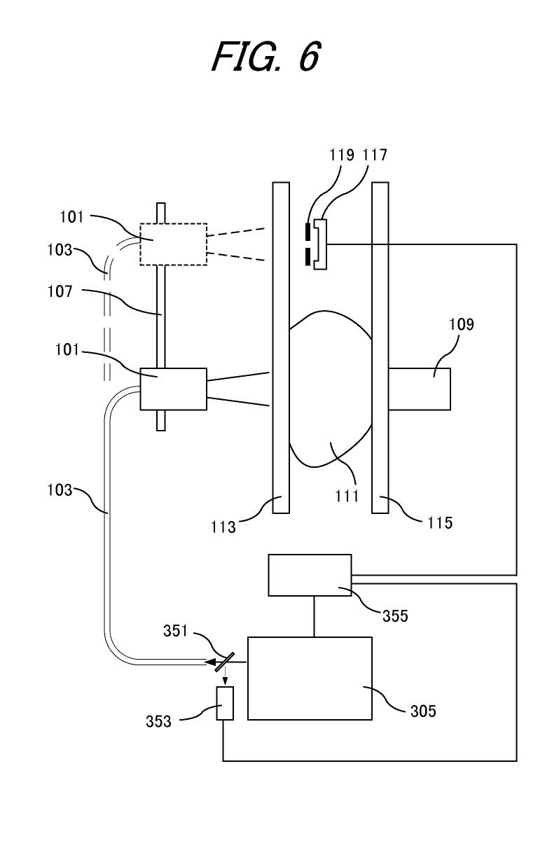

FIG. 6 is a schematic diagram illustrating a fourth example of the present invention. In this diagram, elements identical to those in FIG. 1 are assigned with the identical numerals. The description thereof is omitted.

In this example, a Ti:Sa laser, which is a variable wavelength laser, is adopted as a laser source 305. A part of emitted laser light is taken out by a beam sampler 351, and guided to an optical wavelength meter 353, which is a unit for measuring a wavelength. A unit 355 for controlling optical energy calculates the maximum permissible exposure per pulse based on the repetition rate measured by the optical energy detector 117, a wavelength data measured by the optical wavelength meter 353, and a measurement time preliminarily set by an operator. Further, the unit 355 compares the maximum permissible exposure and the maximum value of the distribution of light fluence measured from the measurement data of the optical energy detector 117 with each other. When the maximum value of the distribution of light fluence exceeds the maximum permissible exposure, the unit 355 controls the laser source 305 such that the output thereof should be equal to or smaller than the maximum permissible exposure. When the maximum value of the distribution of light fluence is smaller than the maximum permissible exposure, the unit 355 causes the laser source 305 to increase the output thereof in an extent of a desired safety factor.

In this example, even in a case where the wavelength control unit included in the Ti:Sa laser has an error, the maximum permissible exposure can optimally be set.

### Example 5

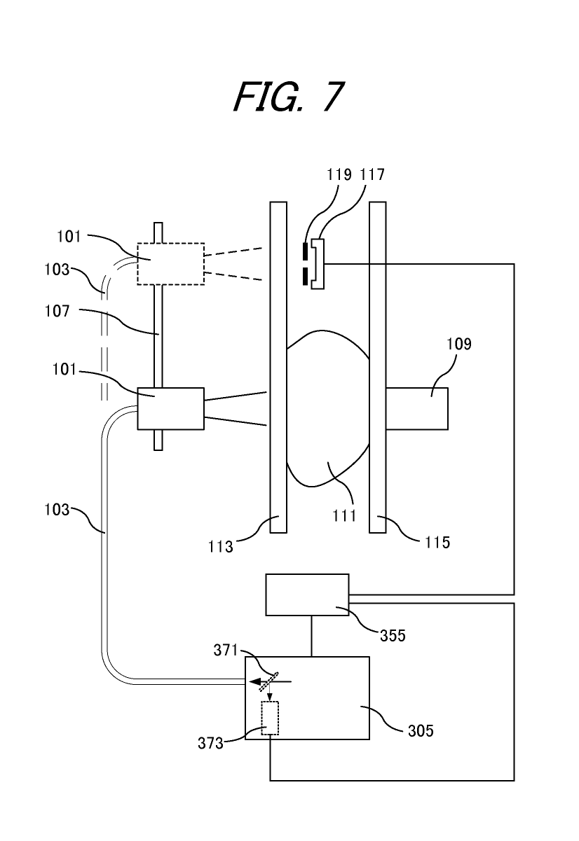

FIG. 7 is a schematic diagram illustrating a fifth example of the present invention. In this diagram, elements identical to those in FIG. 6 are assigned with the identical numerals and descriptions thereof are omitted. This example illustrates a case where a beam sampler 371 and an optical wavelength meter 373 are provided in a casing of a laser source 305 (in the apparatus).

According to this example, a wavelength calibration function can be added to the laser source 305, thereby increasing reliability of information of the tissue to be acquired.

### Example 6

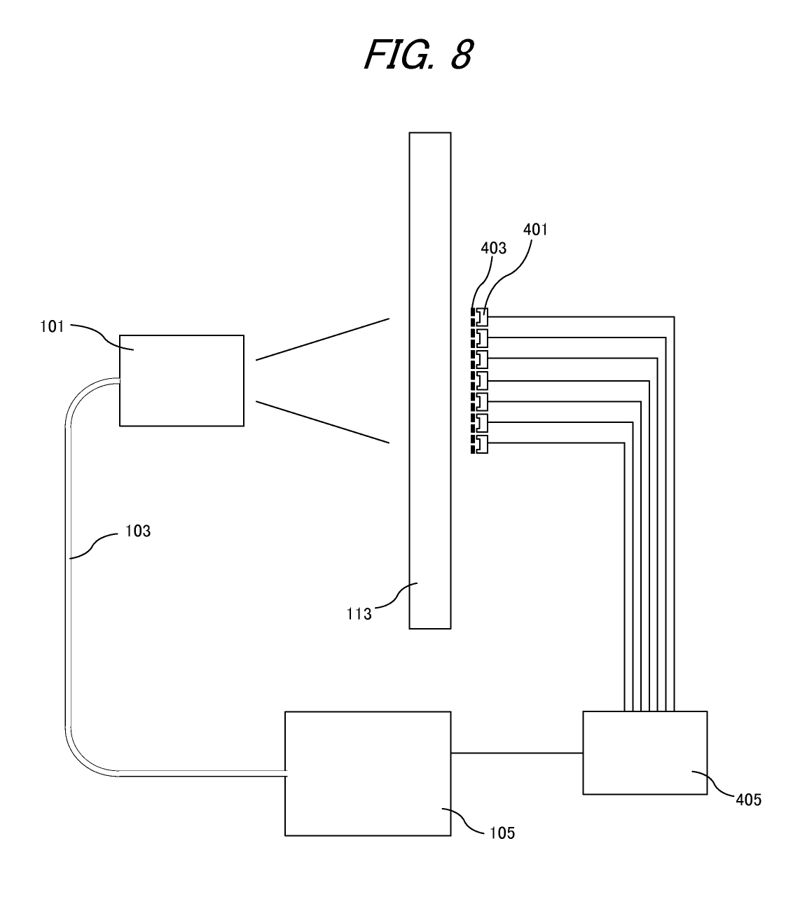

FIG. 8 is a schematic diagram illustrating a sixth example of the present invention. In this diagram, elements identical to those in FIG. 1 are assigned with the identical numerals, and descriptions thereof are omitted. In this example, a unit for detecting optical energy is a group of optical energy detectors including plural optical energy detectors 401 and apertures 403. Information from each optical energy detector 401 is transmitted to a unit 405 for controlling optical energy.

In this example, the optical energy detectors 401 and the aperture 403 may be arranged in series. However, in a case of two-dimensional planar arrangement thereof, the optical energy distribution can be measured without scanning with the unit for light illumination and the optical energy detector. Accordingly, time necessary to measure the optical energy distribution can be reduced.

### Example 7

In cases of implementing the above examples, for some of units for light illumination to be used, there is a problem in that, when the tissue is absent, illumination light is totally reflected by a glass surface of the supporting plate and thereby an exposure amount cannot be measured. In this case, the unit for light illumination capable of causing the illumination light to obliquely propagate through the supporting plate and illuminating a substantially front part of the acoustic wave detector, and the optical part optically matched with the supporting plate are used. Accordingly, the light having obliquely propagated through the supporting plate and applied can be guided into the optical detector.

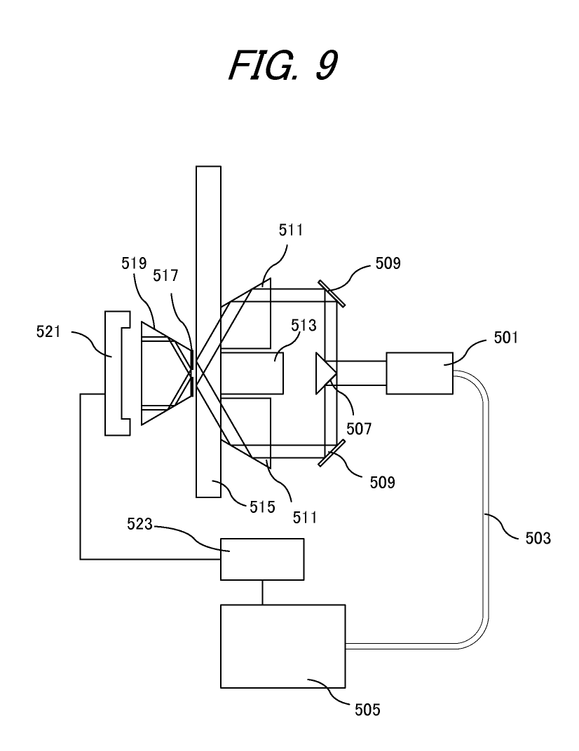

FIG. 9 is a schematic diagram illustrating a seventh example of the present invention. In this example, a unit for light illumination is arranged on a side identical to that of the acoustic wave detector and opposite to the tissue through the supporting plate.

In this diagram, a Nd: YAG laser source 505 has a wavelength of 1064 nm and a pulse width of 10 nsec and a repetition rate of 10 Hz. A unit 503 for light transmission is configured to include optical fibers. The diagram also illustrates a unit 501 for light illumination. Laser light emitted from the unit 501 for light illumination is split into two beams by a branching prism 507, and guided to a substantially front part of an acoustic wave detector 513 via a mirror 509, a reflecting prism 511 and a supporting plate 515. In this case, the laser light obliquely propagates through the supporting plate 515. However, at a certain angle, the light is totally reflected by the interface between the supporting plate 515 and the air, causing a problem in measuring the optical energy. Thus, a coupling prism 519 is arranged such that the coupling prism 519 and the reflecting prism 511 sandwich the supporting plate 515. Provided that the angles of oblique surfaces of the coupling prism 519 and the reflecting prism 511 are adjusted to each other, the light beam is appropriately guided to an optical energy detector 521. The interface between the reflecting prism 511 and the supporting plate 515 or the interface between the coupling prism 519 and the supporting plate 515 can optically contact with each other. Instead, one of water, oil and gel-like liquid may be inserted thereinto as a matching agent.

A surface of the coupling prism 519 contacting with the supporting plate 515 is provided with an aperture 517 with a diameter of 3.5 mm. This configuration is suitable to acquire an light fluence.

The elements 501, 507, 509, 511 and 513 may be integrated and arranged on a driving mechanism capable of two-dimensionally scanning. The optical energy distribution and the distribution of light fluence can be acquired by two-dimensional scanning with this integrated unit.

A unit 523 for controlling optical energy calculates the maximum permissible exposure per pulse, and compares the maximum permissible exposure and the maximum value of the measured distribution of light fluence with each other. When the maximum value of the distribution of light fluence exceeds the maximum permissible exposure, the unit 523 controls the laser source 505 such that the output thereof should be equal to or smaller than the maximum permissible exposure.

In this example, a configuration may be adopted where the optical energy detector 521 and the coupling prism 519 are fixed at positions without interference with holding the tissue. Instead, a detachable configuration may be adopted as with the third example.

While in this example the optical energy distribution is measured by two-dimensionally scanning of the side of the unit for light illumination, a driving mechanism capable of two-dimensionally scanning may be arranged on the side of the optical energy detector.

The optical system for illumination illustrated in this example is only an exemplary case, and the embodiments may not be limited thereto. Any unit capable of illuminating a front part of the acoustic wave detector may be adopted. Further, the shape of coupling prism 519 may not be limited to a trapezoid. Instead, the shape may be determined according to the optical system for illumination. For example, the shape may be one of a cone and a shape of a quadrangular pyramid whose vertex parts are cut out.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2010-103805, filed Apr. 28, 2010, which is hereby incorporated by reference herein in its entirety.

What is claimed is:

1. A measuring apparatus comprising a laser source generating light, a unit for light illumination illuminating tissue with the light, and an acoustic wave detector detecting an acoustic wave generated by the light applied to the tissue, further comprising
a unit for detecting optical energy that detects an light fluence of the light onto the tissue,
wherein an emitted light amount from the laser source is controlled such that the light fluence detected by the unit for detecting optical energy does not exceed a maximum permissible exposure.

2. The measuring apparatus according to claim 1, wherein at least one of the unit for light illumination and the unit for detecting optical energy is provided at a first moving mechanism capable of two-dimensionally moving, and is capable of measuring a distribution of light fluence of the light applied to the tissue by two-dimensional scanning.

3. The measuring apparatus according to claim 1, further comprising a controlling unit that controls the emitted light amount of the laser source such that the light fluence detected by the unit for detecting optical energy does not exceed the maximum permissible exposure.

4. The measuring apparatus according to claim 1,
wherein the unit for detecting optical energy includes a group of optical energy detectors where a plurality of optical energy detectors are arranged in a two-dimensionally planar manner, and
the group of optical energy detectors measures a distribution of light fluence of the light applied to the tissue.

5. The measuring apparatus according to claim 1,
wherein the unit for detecting optical energy is arranged in the apparatus, and
the unit for light illumination is arranged in a second moving mechanism, and capable of switching a state of illuminating the tissue with the light and a state of illuminating the unit for detecting optical energy with the light by moving the second moving mechanism.

6. The measuring apparatus according to claim 5, wherein the second moving mechanism is used commonly with the first moving mechanism.

7. The measuring apparatus according to claim 1, wherein the unit for detecting optical energy is detachable, the detachable unit for detecting optical energy is arranged at a position for holding the tissue, and the light fluence is detected.

8. The measuring apparatus according to claim 3, wherein the unit for detecting optical energy has a function of detecting a repetition rate of illumination light, and the controlling unit controls the emitted light amount from the laser source according to the light fluence and the repetition rate detected by the unit for detecting optical energy.

9. The measuring apparatus according to claim 3, further comprising a unit for measuring wavelength that measures a wavelength of emitted light from the laser source, wherein the controlling unit controls the emitted light amount from the laser source according to the wavelength measured by the unit for measuring wavelength.

10. The measuring apparatus according to claim 9, wherein the unit for measuring wavelength is arranged in a casing of the laser source.

11. The measuring apparatus according to claim 1, wherein the unit for detecting optical energy includes an aperture provided with a window having a size specified by a laser safety standard.

12. The measuring apparatus according to claim 1, further comprising at least one supporting plate contacting with the tissue for holding the tissue.

13. The measuring apparatus according to claim 1, wherein the unit for light illumination and the acoustic wave detector are arranged on a side opposite to the tissue through the supporting plate, the unit for light illumination includes an optical system causing light to propagate obliquely through the supporting plate and illuminating a front of the acoustic wave detector with the light, and the unit for detecting optical energy includes an optical part optically matched with the supporting plate.

14. The measuring apparatus according to claim 13, wherein the optical part is a prism.

15. The measuring apparatus according to claim 13, wherein the optical part is provided with an aperture having a size specified by a laser safety standard on a surface opposed to the supporting plate.
